(19) United States
(12) Patent Application Publication
(10) Pub. No.: US 2012/0134883 A1
(43) Pub. Date: May 31, 2012

(54) Vehicle ventilation deodorizer composition and method

(75) Inventor: Robert Joseph Hecking, Cincinnati, OH (US)

(73) Assignee: KLC Brands, Cincinnati, OH (US)

(21) Appl. No.: 13/200,760

(22) Filed: Sep. 28, 2009

(51) Int. Cl. A61L 9/01; A61L 9/00

(52) U.S. Cl. 422/124; 424/76.1

(57) ABSTRACT

A pressurized vessel with tube to directly propel and insert deodorizer and freshener deep into vehicle ventilation system, to settle particulates and odor causing molecule out of air and onto ventilation system walls where chemical deodorizers can react and neutralize malodors including but not limited to smoke odor from tobacco, food odors from eating in a car, pet odors, mildew odors, body odors, soil, etc. These areas cannot be reached with typical cleaning products or cleaning tools without expensive disassembly. This method provides an economical and practical way to achieve deodorization so important to cars ventilation systems can be deodorized easily. Prior art is to install devices on the outside of the vents—not reaching malodors in system.

### TECHNICAL FIELD

The present invention relates to compositions related to deodorizing the interior of the vehicle ventilation systems and distribute fresh scent when fan in system is turned on for either heat or air conditioning

### BACKGROUND

There are many successful products for the deodorization of the upholstery in these vehicles including pump sprays, aerosols, upholstery extraction, and absorptive powders. Cars and other vehicles are equipped with ventilation systems that supply heat and or air conditioning to the inside of a vehicle. They can operate by drawing air into the vehicle from the outside by invents or by re-circulating the air within the passenger cabin. Vehicle's are designed to cool faster byre-circulating interior air therefore the system is treating mostly interior air and hence a lower volume of air to achieve faster cooling. This recirculation draws air from inside the passenger cabin and also picks up the odors, if the passengers are smoking, eating, have pets in the car, odiferous materials or body odors from sweating.

Current technology does not allow for treatment of odors left in ventilation systems. Some have tried spraying air and fabric fresheners into the invent under the hood, but many cars are equipped with filters at this location, so the products fail effectively reach the source of the malodors in and on the ventilation pipes.

Cars are exposed to many odors that are dragged into the ventilation system, such as smoke, food odors, body odors, etc. U.S. Pat. Des. 347,684 disclosed a device to be permanently installed in the vehicle with a reservoir (for scent and/or deodorizer base) and a nozzle, but does not delineate the direction of the nozzle tube. It appears it is released in the cabin and picked up by the intake in the cabin. This device if installed could be tampered with and could release scent into the face of the driver while driving presenting a hazard. Our embodiment allows dispensing odor neutralizer into the ventilation system, knocking vapors down onto ventilation system walls and odor neutralization in the systems, not just the air exiting the system. The deodorizing action is completed and scent release is minimal not jeopardizing the operation of the vehicle.

U.S. Pat. No. 3,006,042 is a device that is plugged into the cigarette lighter, using the car's electrical service to power the release of scent or deodorizer. This does not address bad odors in the ventilation system.

U.S. Pat. No. 1,311,978 is for a plastic device to be attached to the exterior of the vents in the car hence fragrancing air, but not able to deodorize the interior of the ventilation system

Spraying perfumes and deodorizers directly onto car dashboard can damage sensitive plastic surfaces, stainless steel appearance, or react with plastic hinges on vent direction louvers. Our invention has been tested with ventilation materials, and reaches past surface of dashboard trim to reach deep into ventilation system and deodorize and freshen air.

### SUMMARY

Our invention is a vessel either aerosol can, pump spray or pressurized reservoir to be driven by compressed air driven through the valve into a six-inch tube with 1/32″ inside diameter to deliver product into ventilation system. A pressurized container using a nozzle with the six inch tube is also claimed,

Basically, the present invention provides a proprietary deodorizer and scent that by the power of aerosol gases or compressed gas can be inserted into air intakes, air vents and defroster outlets or inlets of the vehicle and reach interior ventilation system, and provide deodorized fresh air to the renter.

### BRIEF DESCRIPTION OF THE DRAWINGS

FIG. 1 shows a cutaway view of an embodiment of the ventilation system in an automobile utilized in the process of the present invention.

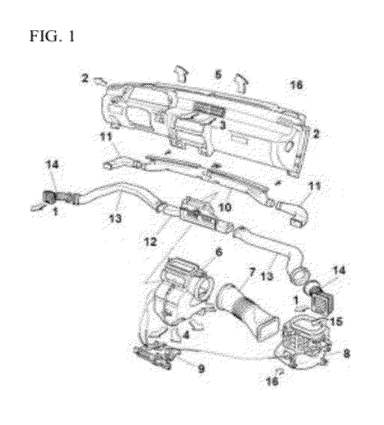

### DETAILED DESCRIPTION

The present invention provides a method (a pressure propelled gas of glycol based product to precipitate odors on ventilation systems walls with a deodorizer to neutralize odors a scent to freshen, through a small diameter tube to reach into ventilation systems were odors can be trapped. The invention can deliver and dispense a freshening agent (e.g. fragrance, perfume, deodorant, absorbent, odorant, perfume, deodorizer, or combination of any two) to reach malodors and deodorize them in vehicle ventilation systems economically.

The formula must not affect the plastics, fabrics on ventilation tubes, must comply with V.O.C. (Volatile Organic Compounds) regulations.

What is claimed is:

1. A non-cleaning, vehicle ventilation deodorizing product, comprising:
a pressurized product comprised of a deodorizer and freshening scent product in a solution of water, glycol and alcohol mixture;
(either aerosol or mechanical pump driven) with direct insertion tube.

2. The non-cleaning formula according to claim 1, further comprising a glycol base chemical to drag odiferous malodors inside the ventilation pipes/system and settles the mixture on ventilation systems tubes, pipes and or filters where odors can be neutralized and fresh scent (any perfume) is then dispensed when ventilation fan is turned on. The embodiments of the patent search either treat the air as it come out, and do not deodorize interior of system, or are complex machines used to release scents into car cabin air, again without deodorizing interior of ventilation system
